SEQUENCE LISTING (27 sequences)

SEQ ID NO: 1 - protein, 114 aa, homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSL

SEQ ID NO: 2 - protein, 109 aa, homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLL

SEQ ID NO: 3 - protein, 104 aa, Homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEF

SEQ ID NO: 4 - protein, 99 aa, Homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIE

SEQ ID NO: 5 - protein, 60 aa, Homo sapiens
EDASSDSTGADPAQEAGTSKPNEEISGPAEPASPPETTTTAQETSAAAVQGTAKVTSSRQ

SEQ ID NO: 6 - protein, 114 aa, Homo sapiens
DSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 7 - protein, 109 aa, Homo sapiens
DPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 8 - protein, 104 aa, Homo sapiens
AGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 9 - protein, 98 aa, Homo sapiens
NEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 10 - protein, 77 aa, Homo sapiens
ETSAAAVQGTAKVTSSRQELNPLKSIVEKSILLTEQALAKAGKGMHGGVPGGKQFIENGSEFAQKLLKKFSLLKPWA

SEQ ID NO: 11 - protein, 61 aa, Homo sapiens
MQELNPLKSIVEKSILLTEQALAKAGKGMHGGVPGGKQFIENGSEFAQKLLKKFSLLKPWA

SEQ ID NO: 12 - protein, 85 aa, Homo sapiens
METTTTAQETSAAAVQGTAKVTSSRQELNPLKSIVEKSILLTEQALAKAGKGMHGGVPGGKQFIENGSEFAQKLLKKFSLLKPWA

SEQ ID NO: 13 - protein, 75 aa, Homo sapiens
MAAAVQGTAKVTSSRQELNPLKSIVEKSILLTEQALAKAGKGMHGGVPGGKQFIENGSEFAQKLLKKFSLLKPWA

SEQ ID NO: 14 - protein, 55 aa, Homo sapiens
MKSIVEKSILLTEQALAKAGKGMHGGVPGGKQFIENGSEFAQKLLKKFSLLKPWA

SEQ ID NO: 15 - protein, 49 aa, Homo sapiens
MSILLTEQALAKAGKGMHGGVPGGKQFIENGSEFAQKLLKKFSLLKPWA

SEQ ID NO: 16 - protein, 120 aa, Homo sapiens
MEDASSDSTGADPAQEAGTSKPNAGISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 17 - protein, 120 aa, Homo sapiens
MEDASSDSTGADPAQEAGTSKPNEEISGPAEPASPPETTTTAQETSAAAVQGTAKVTSSRQELNPLKSIVEKSILLTEQALAIAGIGMHGGVPGGKQFIENGSEFAQKLLKKFSLLKPWA

SEQ ID NO: 18 - protein, 120 aa, Homo sapiens
MEDASSDSTGADPAQEAGTSKPNAGISGPAEPASPPETTTTAQETSAAAVQGTAKVTSSRQELNPLKSIVEKSILLTEQALAIAGIGMHGGVPGGKQFIENGSEFAQKLLKKFSLLKPWA

SEQ ID NO: 19 - protein, 123 aa, Homo sapiens
YYYYEDASSDSTGADPAQEAGTSKPNEEISGPAEPASPPETTTTAQETSAAAVQGTAKVTSSRQELNPLKSIVEKSILLTEQALAIAGIGMHGGVPGGKQFIENGSEFAQKLLKKFSLLKPWA

SEQ ID NO: 20 - protein, 118 aa, Homo sapiens
YYYYEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSL

SEQ ID NO: 21 - protein, 113 aa, Homo sapiens
YYYYEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLL

SEQ ID NO: 22 - protein, 108 aa, Homo sapiens
YYYYEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEF

SEQ ID NO: 23 - protein, 103 aa, Homo sapiens
YYYYEDASSDSTGADPAQEAGTSKPNEEISGPAEPASPPETTTTAQETSAAAVQGTAKVTSSRQELNPLKSIVEKSILLTEQALAKAGKGMHGGVPGGKQFIE

SEQ ID NO: 24 - protein, 98 aa, Homo sapiens
YYYYEDASSDSTGADPAQEAGTSKPNEEISGPAEPASPPETTTTAQETSAAAVQGTAKVTSSRQELNPLKSIVEKSILLTEQALAKAGKGMHGGVPGG

SEQ ID NO: 25 - protein, 64 aa, Homo sapiens
YYYYEDASSDSTGADPAQEAGTSKPNEEISGPAEPASPPETTTTAQETSAAAVQGTAKVTSSRQ

SEQ ID NO: 26 - protein, 138 aa, Homo sapiens
MKFTTLLFLAAVAGALVYAEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 27 - protein, 119 aa, Homo sapiens
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

(19) United States
(12) Patent Application Publication
(10) Pub. No.: US 2009/0312252 A1
(43) Pub. Date: Dec. 17, 2009

(54) Antimicrobial Activity in Variants of Lacritin

(75) Inventors: Robert L. McKown, Bridgewater, VA (US); Ronald W. Raab, Harrisonburg, VA (US); Gordon W. Laurie, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/441,178

(22) PCT Filed: Sep. 13, 2007
(86) PCT No.: PCT/US07/19957

§ 371 Date: Mar. 13, 2009

Related U.S. Application Data
(60) Provisional application No. 60/844,353, filed on Sep. 14, 2006.

(51) Int. Cl. A61K 38/00; A61K 31/7088

(52) U.S. Cl. 514/12; 514/44 R

(57) ABSTRACT

The present invention provides recombinant proteins with antimicrobial activity and methods for treating animals including humans by administering the novel recombinant proteins. In particular, the invention provides methods for treating and/or preventing microbial diseases and infections using lacritin and homologs, fragments, modifications, and derivatives thereof.

### CROSS REFERENCE TO RELATED APPLICATIONS

This application is entitled to priority pursuant to 35 U.S.C. §119(e) to U.S. provisional patent application No. 60/844,353 filed on Sep. 14, 2006.

### STATEMENT REGARDING FEDERALLY SPONSORED RESEARCH OR DEVELOPMENT

This invention was supported in part from Grant No. R01EY13143 awarded by the National Institutes of Health. The United States Government has certain rights in the invention.

### FIELD OF THE INVENTION

The present invention is directed generally to the field of molecular genetics and to antimicrobial agents. More particularly, the present invention describes novel recombinant proteins and novel methods for treating animals, including humans, by administering the novel recombinant proteins.

### BACKGROUND

Human pathogenic microbes are increasingly becoming resistant to established antibiotic drugs approved for human use, thereby creating a need for new antimicrobial drugs. Only three new structural classes of antibiotics have been introduced into medical practice in the past 40 years and certain pathogenic bacteria have become resistant to all these classes. Moreover, all antimicrobial drugs on the market have some relative degree of host toxicity that is concentration dependent.

Recombinant antimicrobial proteins represent a new class of antimicrobial drugs for the treatment of human diseases that do not respond to established drugs. In particular, recombinant proteins derived from naturally occurring human proteins can be used as antimicrobials without the fear of toxic side effects to the human patients.

Lacritin was discovered as a novel secretion enhancing factor from the human lacrimal gland (Sanghi et al., J. Mol. Biol. 2001 Jun. 29;310(1):127-39). Mature Lacritin is a human tear protein composed of 119 amino acids that is secreted by the lacrimal gland. It has even been shown to be expressed in breast cancer cells (Weigelt et al., J. Cancer Res. Clin. Oncol. 2003 December;129(12):735-6). It has been previously shown that recombinant lacritin produced in bacteria promotes new cell growth in cultured human salivary gland cells and increases tear production with topical application to the eyes of rabbits (A. R. Spitze, et al., Extended Treatment with Lacritin, a Novel Tear Glycoprotein, Stimulates Tear Production in Rabbits, 2006 ARVO Annual Meeting). Lacritin signals to STIM1, mTOR and NFATC1 via rapid PKCA dephosphorylation and PLD activation to potentially regulate differentiation, renewal and secretion by the non-germative exocrine epithelia that it preferentially targets (Wang et al., J. Cell Biol. 2006 Aug. 28;174(5):689-700).

There is a long felt need in the art for new antimicrobial compounds and methods for treating microbial infections. The present invention satisfies these needs.

### SUMMARY OF THE INVENTION

The present invention is based on the discovery disclosed herein that lacritin and fragments, homologs, derivatives, and modifications thereof have antimicrobial activity. The present invention provides, inter alia, novel lacritin fragments which maintain the activity(s) of lacritin. In one aspect, the activity is antimicrobial activity. In one aspect, the antimicrobial activity is bactericidal activity. In another aspect, the antimicrobial activity is bacteriostatic activity.

In one aspect, the variants are homologs, fragments, derivatives, and/or modifications of native lacritin or mature lacritin. Native lacritin (also called lacritin precursor) is a 138 amino acid residue polypeptide (NCBI accession numbers NP_{—}150953 and Q9GZZ8) having the sequence:

(SEQ ID NO: 26)

MKFTTLLFLAAVAGALVYAEDASSDSTGADPAQEAGTSKPNEEI
SGPAEPASPPETTTTAQETSAAAVQGTAKVTSSRQELNPLKSIVEKSILL
TEQALAKAGKGMHGGVPGGKQFIENGSEFAQKLLKKFSLLKPWA.

Mature lacritin is a 119 amino acid residue polypeptide having the amino acid sequence:

(SEQ ID NO: 27)

EDASSDSTGADPAQEAGTSKPNEEISGPAEPASPPETTTTAQETSAAAV
QGTAKVTSSRQELNPLKSIVEKSILLTEQALAKAGKGMHGGVPGGKQFIE
NGSEFAQKLLKKFSLLKPWA.

In some embodiments, the antimicrobial mature lacritin protein (SEQ ID NO:27) and variants thereof comprise proteolytically cleaved lacritin specific for the pathogen that produces the protease. In some embodiments, the invention provides amphipathic antimicrobial lacritin protein variants. In further embodiments, the novel recombinant lacritin protein variant comprises an amino acid sequence selected from the group consisting of SEQ ID NOs: 1-27. In one aspect, polypeptides comprising SEQ ID NOs:1-25 are used.

The invention provides compositions and methods comprising the use of lacritin and fragments, homologs, derivatives, and modifications thereof as antimicrobial agents. In one aspect, the antimicrobial activity is antibacterial activity. In one aspect, an effective amount of lacritin and fragments, homologs, derivatives, and modifications thereof is about 0.01 to about 100.0 μg/ml. In one aspect, an effective amount is about 0.1 to about 50 μg/ml. In yet another aspect, an effective is about 0.5 to about 11 μg/ml. In one aspect, the homologs comprise up to about 10 amino acid substitutions. In one aspect, the substitutions are conservative. In another aspect, the homologs comprise up to about 5 amino acid substitutions of lacritin and fragments, homologs, derivatives, and modifications thereof.

The invention also provides compositions and methods for treating or preventing microbial diseases and infections. In one aspect, the infection is a bacterial infection. In one aspect, the bacteria are selected from the group consisting of Escherichia coli, Pseudomonas aeruginosa, Staphylococcus aureus, and Staphylococcus epidermidis.

In some embodiments, the methods comprise administering to a subject a therapeutically effective amount of at least one antimicrobial lacritin protein or variant thereof. In one aspect, the at least one protein variant comprises an amino acid sequence selected from the group consisting of SEQ ID NOs: 1-25.

In one aspect, the invention encompasses a composition for treating or preventing an infection, said composition comprising a therapeutically effective amount of at least one purified polypeptide comprising the amino acid sequence of a polypeptide of the invention, or a fragment, homolog, modification, or derivative thereof, a pharmaceutically acceptable carrier, and optionally a purified antimicrobial agent. In one aspect, the polypeptide has a sequence selected from the group consisting of SEQ ID NOs:1 -27, or a fragment, homolog, modification, or derivative thereof. In one aspect, the additional antimicrobial agent is selected from the group consisting of antibiotics, antimycotics, benzalkonium chloride, benzethonium chloride, benzyl alcohol, chlorobutanol, chlorhexidine digluconate or diacetate, methyl and propyl hydroxybenzoate (parabens), phenylethyl alcohol, phenylmercuric acetate or nitrate, sorbic acid, and thimerosal.

In another embodiment, the invention encompasses a composition for preventing or treating an infection, said composition comprising a therapeutically effective amount of an isolated nucleic acid comprising a nucleic acid sequence encoding a polypeptide of the invention, or a fragment, or homolog thereof, a pharmaceutically acceptable carrier, and a purified antimicrobial agent.

In one aspect, the composition comprises a topical formulation. In another aspect, the composition further comprises a pharmaceutically acceptable phospholipid or oil.

The invention further provides pharmaceutical compositions comprising at least one antimicrobial lacritin protein variant. In some embodiments, the pharmaceutical compositions further comprise at least one non-lacritin antimicrobial agent or antibiotic. In some embodiments, the pharmaceutical compositions are formulated for topical, oral, nasal, subcutaneous, direct, or parenteral administration. In further embodiments, the at least one protein variant comprises an amino acid sequence selected from the group consisting of SEQ ID NOs: 1-27.

The invention further provides administering isolated nucleic acids comprising nucleic acid sequences encoding the polypeptides and peptides of the invention.

The invention additionally provides kits comprising at least one antimicrobial lacritin protein variant, disposed in a suitable container. In some embodiments, the kits further comprise at least one antimicrobial agent or antibiotic. In further embodiments, the at least one protein variant comprises an amino acid sequence selected from the group consisting of SEQ ID NOs: 1-25.

Various aspects and embodiments of the invention are described in further detail below.

### BRIEF DESCRIPTION OF THE DRAWINGS

FIG. 1 schematically illustrates an example of a vector used to clone and express recombinant variants of lacritin in bacterial cell culture expression systems. Variant lacritin DNA sequences were created with standard molecular biology techniques and cloned in-frame with the intein gene either at the C-terminus or N-terminus. Primer binding sites within the vector were used to sequence all constructs as well as the cloning junction sites.

FIG. 2 illustrates an example of expression of a recombinant intein-lacritin fusion protein 5 hours after induction with IPTG in bacterial cell culture. Samples were visualized by SDS Polyacrylamide Gel Electrophoresis stained with Coomassie blue.

FIG. 3 illustrates an example of purified variant lacritin proteins. The variants shown are (lane number in parentheses): (1) pLAC, (2) C-5, (3) C-10, (4) C-15, (5) C-20, (6) C-25. Samples were visualized by SDS Polyacrylamide Gel Electrophoresis stained with Coomassie blue.

FIGS. 4 and 5 illustrate examples of antimicrobial experiments in which a number of different lacritin variant proteins were incubated with the bacteria E. coli and then plated for colony forming units.

FIG. 6 illustrates SDS Polyacrylamide Gel Electrophoresis of Lacritin Proteins. A. Purification of mature lacritin. Lanes (1) molecular weight markers labeled in kilodaltons (2) fraction 1, cleared cell lysate (3) fraction 2, chitin purified (4) fraction 3, DEAE purified. B. DEAE purified lacritin proteins. Lanes (1) molecular weight markers labeled in kilodaltons (2) mature lacritin (3) N-35 (4) N-45 (5) N-55 (6) N-65 (7) N-71 (8) N-72 (9) N-73 (10) N-75. N-XX denotes the number of amino acids removed from the N-terminal of mature lacritin. 15% acrylamide gels from BioRad were run at 140 volts and silver stained.

FIG. 7 illustrates Antimicrobial activity of selected lacritin constructs. pLAC is full length mature lacritin without signal peptide. N-XX denotes the number of amino acids removed from the N-terminal of mature lacritin and C-XX denotes the number of amino acids removed from the C-terminal of mature lacritin. The numbers 0 through 119 are the amino acid residues of mature lacritin from the C-terminus to the N-terminus.

FIG. 8 illustrates Antimicrobial activity of lacritin N-55. Increasing concentrations of the purified protein were incubated with E. coli in 10 mM phosphate buffer for 3 hours as described in the antimicrobial assay. The total number of colonies were counted and the percent of cell death determined using [1-(colonies surviving peptide incubation)/(colony count from PBS control)]×100.

FIG. 9 graphically illustrates a time course of antimicrobial activity of Lacritin. Lacritin construct N-55 (▪ 30 μg/ml), mature Lacritin (▴ 30 μg/ml) and the phosphate buffered saline control () were incubated with E. coli from 0 to 3 hours as described in the antimicrobial assay. Colonies were counted and the percent cell death was determined using [1-(cells surviving peptide)/(average colonies counted from PBS control)]×100%.

FIG. 10 graphically illustrates the stability of lacritin N-55 antimicrobial activity. Increasing concentrations of the purified protein were incubated with E. coli in 10 mM phosphate buffer for 3 hours as described in the antimicrobial assay. Aliquots of lacritin were stored at −70 degrees C. and assayed after one week and one month of storage as shown. The total number of colonies were counted and the percent of cell death determined using [1-(colonies surviving peptide incubation)/(colony count from PBS control)]×100.

FIG. 11 graphically illustrates Inner Membrane Permeabilization of E. coli ML-35. Mature Lacritin (pLac) and various constructs of Lacritin were incubated with permease-deficient E. coli ML-35 at room temperature for 18 hours. The conversion of ONPG (o-nitrophenyl-β-D-galactopyranoside) to ONP (o-nitrophenyl) by cytoplasmic β-galactosidase was measured by a spectrophotometer at 415 nm. The concentrations of each protein were normalized to 30 μg/ml. This enzymatic assay indicates that both the mature antimicrobial peptide Lacritin and the construct N-55 damage the cell membrane in such a way that cytoplasmic β-galactosidase is able to react with the surrounding ONPG substrate.

### DETAILED DESCRIPTION

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

A disease or disorder is “alleviated” if the severity of a symptom of the disease, condition, or disorder, or the frequency with which such a symptom is experienced by a subject, or both, are reduced.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

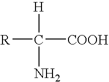

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “another antimicrobial agent”, as used herein, refers to an antimicrobial agent other than a lacritin polypeptide, or a fragment, homolog, derivative, or modification thereof.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies.

“Antimicrobial”, as used herein, refers to a substance, compound, or agent that kills or slows the growth of microbes, such as bacteria, fungi, viruses, or parasites.

The term “antimicrobial agent”, as used herein, refers to a compound or agent with the ability to impede the growth of a microbe. Impeding growth further includes an agent which kills the microbe. For example, various antimicrobial agents act, inter alia, by interfering with (1) cell wall synthesis, (2) plasma membrane integrity, (3) nucleic acid synthesis, (4) ribosomal function, and (5) folate synthesis. One of ordinary skill in the art will appreciate that a number of “antimicrobial susceptibility” tests can be used to determine the efficacy of a candidate antimicrobial agent.

The term “antibacterial agent”, as used herein, refers to a compound or agent with the ability to kill or impede the growth of bacteria.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

As used herein, the term “biologically active fragments” or “bioactive fragment” of the polypeptides encompasses natural or synthetic portions of the full-length protein that are capable of specific binding to their natural ligand or of performing the function of the protein.

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

The term “competitive sequence” refers to a peptide or a modification, fragment, derivative, or homolog thereof that competes with another peptide for its cognate binding site.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

As used herein, a “derivative” of a compound refers to a chemical compound that may be produced from another compound of similar structure in one or more steps, as in replacement of H by an alkyl, acyl, or amino group.

The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence polarization or altered light scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA). or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein. A fragment of a lacritin peptide which is used herein as part of a composition for use in a treatment or to elicit a lacritin effect, is presumed to be a biologically active fragment for the response to be elicited.

As used herein, a “functional” biological molecule is a biological molecule in a form in which it exhibits a property or activity by which it is characterized. A functional enzyme, for example, is one which exhibits the characteristic catalytic activity by which the enzyme is characterized.

The terms “formula” and “structure” are used interchangeably herein.

As used herein, a “gene” refers to the nucleic acid coding sequence as well as the regulatory elements necessary for the DNA sequence to be transcribed into messenger RNA (mRNA) and then translated into a sequence of amino acids characteristic of a specific polypeptide.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “inhibit,” as used herein, refers to the ability of a compound or any agent to reduce or impede a described function or pathway. Preferably, inhibition is by at least 10%, more preferably by at least 25%, even more preferably by at least 50%, and most preferably, the function is inhibited by at least 75%.

The term “inhibit microbial growth”, as used herein, refers to inhibiting growth or to killing a microbe.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, the term “insult” refers to contact with a substance or environmental change that results in an alteration of normal cellular metabolism in a cell or population of cells. Environmental insults may include, but are not limited to, chemicals, environmental pollutants, heavy metals, microbial infections, changes in temperature, changes in pH, as well as agents producing oxidative damage, DNA damage, or pathogenesis. The term “insult” is used interchangeably with “environmental insult” herein.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, the term “lacritin polypeptide” and like terms refers to peptides comprising the amino acid sequence of the full length lacritin and biologically active fragments, derivatives, modifications, and homologs thereof. As used herein, the term “biologically active fragments” or “bioactive fragment” of a lacritin polypeptide encompasses natural or synthetic portions of the amino acid sequence of the full length peptide.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

A “marker” is an atom or molecule that permits the specific detection of a molecule comprising that marker in the presence of similar molecules without such a marker. Markers include, for example radioactive isotopes, antigenic determinants, nucleic acids available for hybridization, chromophors, fluorophors, chemiluminescent molecules, electrochemically detectable molecules, molecules that provide for altered fluorescence-polarization or altered light-scattering and molecules that allow for enhanced survival of an cell or organism (i.e. a selectable marker). A reporter gene is a gene that encodes for a marker.

The term “modulate”, as used herein, refers to changing the level of an activity, function, or process. The term “modulate” encompasses both inhibiting and stimulating an activity, function, or process.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

“Ocular surface,” as used herein, refers to the surface of the eye, particularly the corneal surface.

“Operably linked” refers to a juxtaposition wherein the components are configured so as to perform their usual function. Thus, control sequences or promoters operably linked to a coding sequence are capable of effecting the expression of the coding sequence.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A “polylinker” is a nucleic acid sequence that comprises a series of three or more different restriction endonuclease recognitions sequences closely spaced to one another (i.e. less than 10 nucleotides between each site).

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH2-carbamate linkage (—CH2OC(O)NR—), a phosphonate linkage, a —CH2-sulfonamide (—CH2-S(O)2NR—) linkage, a urea (—NHC(O)NH—) linkage, a —CH2-secondary amine linkage, or with an alkylated peptidyl linkage (—C(O)NR—) wherein R is C1-C4 alkyl;

2. peptides wherein the N-terminus is derivatized to a —NRR1 group, to a —NRC(O)R group, to a —NRC(O)OR group, to a —NRS(O)2R group, to a —NHC(O)NHR group where R and R1 are hydrogen or C1-C4 alkyl with the proviso that R and R1 are not both hydrogen;

3. peptides wherein the C terminus is derivatized to —C(O)R2 where R2 is selected from the group consisting of C1-C4 alkoxy, and —NR3R4 where R3 and R4 are independently selected from the group consisting of hydrogen and C1-C4 alkyl.

Synthetic or non-naturally occurring amino acids refer to amino acids which do not naturally occur in vivo but which, nevertheless,.can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A “promoter” is a DNA sequence that directs the transcription of a DNA sequence, such as the nucleic acid coding sequence of a gene. Typically, a promoter is located in the 5′ region of a gene, proximal to the transcriptional start site of a structural gene. Promoters can be inducible (the rate of transcription changes in response to a specific agent), tissue specific (expressed only in some tissues), temporal specific (expressed only at certain times) or constitutive (expressed in all tissues and at a constant rate of transcription).

A “core promoter” contains essential nucleotide sequences for promoter function, including the TATA box and start of transcription. By this definition, a core promoter may or may not have detectable activity in the absence of specific sequences that enhance the activity or confer tissue specific activity.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, the term “purified” and like terms relate to the isolation of a molecule or compound in a form that is substantially free of contaminants normally associated with the molecule or compound in a native or natural environment. The term “purified” does not necessarily indicate that complete purity of the particular molecule has been achieved during the process. A “highly purified” compound as used herein refers to a compound that is greater than 90% pure.

A “subject” of experimentation, diagnosis or treatment is an animal, including a human.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “substantially pure nucleic acid”, as used herein, refers to a nucleic acid sequence, segment, or fragment which has been purified from the sequences which flank it in a naturally occurring state, e.g., a DNA fragment which has been removed from the sequences which are normally adjacent to the fragment e.g., the sequences adjacent to the fragment in a genome in which it naturally occurs. The term also applies to nucleic acids which have been substantially purified from other components which naturally accompany the nucleic acid, e.g., RNA or DNA or proteins which naturally accompany it in the cell.

A “therapeutic agent” is one used to treat a disease or disorder.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “treating” includes prophylaxis of the specific infection, disorder or condition, or alleviation of the symptoms associated with a specific infection, disorder or condition and/or preventing or eliminating said symptoms. A “prophylactic” treatment is a treatment administered to a subject who does not exhibit signs of an infection or disease or exhibits only early signs of the disease for the purpose of decreasing the risk of developing pathology associated with the disease. As used herein, the term “treating” includes alleviating the symptoms associated with a specific infection, disease, disorder or condition and/or preventing or eliminating said symptoms.

A “vector” is also meant to include a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, plasmids, cosmids, lambda phage vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

As used herein, the term “wound” relates to a physical tear or rupture to a tissue or cell layer. A wound may occur by any physical insult, including a surgical procedure.

Techniques and information included in U.S. Pat. Pub. 2004/0081984 (Laurie et al., published Apr. 29, 2004), WO 2005/119899 (Laurie et al.; published Dec. 15, 2005), and WO 98/27205 (published Jun. 25, 1998) are also useful in the present invention and WO 2005/119899 are hereby incorporated by reference in its entirety herein.

### Embodiments

For the purposes of promoting an understanding of the principles of the invention, reference will now be made to specific embodiment and specific language will be used to describe the same. It will nevertheless be understood that no limitation of the scope of the invention is thereby intended, such alteration and further modifications of the invention, and such further applications of the principles of the invention as illustrated herein, being contemplated as would normally occur to one skilled in the art to which the invention relates.

All terms as used herein are defined according to the ordinary meanings they have acquired in the art. Such definitions can be found in any technical dictionary or reference known to the skilled artisan, such as the McGraw-Hill Dictionary of Scientific and Technical Terms (McGraw-Hill, Inc.), Molecular Cloning: A Laboratory Manual (Cold Springs Harbor, N.Y.), and Remington's Pharmaceutical Sciences (Mack Publishing, Pa.). These references, along with those references and patents cited herein are hereby incorporated by reference in their entirety.

Full-length native lacritin is a human tear protein that is known to have mitogenic activity. The inventors have surprisingly discovered that novel recombinant variants of native human lacritin have biological activities unrelated to mitogenic effects. In particular, novel recombinant lacritin variants genetically engineered by the inventors were shown to have antimicrobial activity.

Thus, in some aspects the present invention is directed to novel recombinant lacritin protein variants with antimicrobial activity. “Antimicrobial” as used herein refers to a substance that kills or slows the growth of microbes, such as bacteria, fungi, viruses, or parasites. A “variant” of lacritin protein, as used herein, refers to a polypeptide with an amino acid sequence that exhibits substantial homology with the amino acid sequence of mature human lacritin (SEQ ID NO:27), or is a fragment, derivative, or modification thereof. The variant may be arrived at by modification of the native amino acid sequence by such modifications as insertion, substitution or deletion of one or more amino acids, or it may be a naturally occurring variant.

The present invention provides, inter alia, novel lacritin protein fragments, and compositions and methods for using lacritin proteins and novel lacritin protein fragments. Additionally, the present invention provides methods for preparing and testing such variants and other methods are also available and known to those of ordinary skill in the art.

Non-limiting examples of some of the recombinant lacritin protein variants provided by the invention include the protein variants of Table 1. N-XX denotes the number of amino acids removed from the N-terminal of mature lacritin and C-XX denotes the number of amino acids removed from the C-terminal of mature lacritin. For the peptides having the sequences of SEQ ID NOs:16-18, the one letter amino acid abbreviations with subscripts denote the amino acid residue number from the N-terminus of mature lacritin (Table 1). The arrows represent an amino acid substitution at that residue number and point to the letter indicating the new amino acid being used as the substitute. For SEQ ID NOs:19-25, “4Y” denotes the addition of 4 tyrosine amino acid residues at the N-terminus of the indicated polypeptide or fragment of mature lacritin.

TABLE 1

Variants of Mature (SEQ ID NO: 27) Lacritin and
Other useful Peptides

| Name of | SEQ ID | |
|---|---|---|
| Construct | NO. | Description/Comments |
| C-5 | 1 | EDASSDSTGADPAQEAGTSKPNFEISGPAEPASP |
| | | PETTTTAQETSAAAVQGTAKVTSSRQELNPLKS |
| | | IVEKSILLTEQALAKAGKGMHGGVPGGKQFIEN |
| | | GSEFAQKLLKKFSL |
| C-10 | 2 | EDASSDSTGADPAQEAGTSKPNEEISGPAEPASP |
| | | PETTTTAQETSAAAVQGTAKVTSSRQELNPLKS |
| | | IVEKSILLTEQALAKAGKGMHGGVPGGKQFIEN |
| | | GSEFAQKLL |
| C-15 | 3 | EDASSDSTGADPAQEAGTSKPNEEISGPAEPASP |
| | | PETTTTAQETSAAAVQGTAKVTSSRQELNPLKS |
| | | IVEKSILLTEQALAKAGKGMHGGVPGGKQFIEN |
| | | GSEF |
| C-20 | 4 | EDASSDSTGADPAQEAGTSKPNEEISGPAEPASP |
| | | PETTTTAQETSAAAVQGTAKVTSSRQELNPLKS |
| | | IVEKSILLTEQALAKAGKGMHGGVPGGKQFIE |
| C-59 | 5 | EDASSDSTGADPAQEAGTSKPNEEISGPAEPASP |
| | | PETTYTAQETSAAAVQGTAKVTSSRQ |
| N-5 | 6 | DSTGADPAQEAGTSKPNEEISGPAEPASPPETTT |
| | | TAQETSAAAVQGTAKVTSSRQELNPLKSIVEKS |
| | | ILLTEQALAKAGKGMHGGVPGGKQFIENGSEF |
| | | AQKLLKKFSLLKPWA |
| N-10 | 7 | DPAQEAGTSKPNEEISGPAEPASPPETTTTAQET |
| | | SAAAVQGTAKVTSSRQELNPLKSIVEKSILLTE |
| | | QALAKAGKGMHGGVPGGKQFIENGSEFAQKL |
| | | LKKFSLLKPWA |
| N-15 | 8 | AGTSKPNEEISGPAEPASPPETTTTAQETSAAAV |
| | | QGTAKVTSSRQELNPLKSIVEKSILLTEQALAK |
| | | AGKGMHGGVPGGKQFIENGSEFAQKLLKKFSL |
| | | LKPWA |
| N-21 | 9 | NEEISGPAEPASPPETTTTAQETSAAAVQGTAK |
| | | VTSSRQELNPLKSIVEKSILLTEQALAKAGKGM |
| | | HGGVPGGKQFIENGSEFAQKLLKKFSLLKPWA |
| N-42 | 10 | ETSAAAVQGTAKVTSSRQELNPLKSIVEKSILLT |
| | | EQALAKAGKGMHGGVPGGKQFIENGSEFAQK |
| | | LLKKFSLLKPWA |
| N-59 | 11 | MQELNPLKSIVEKSILLTEQALAKAGKGMHGG |
| | | VPGGKQFIENGSEFAQKLLKKFSLLKPWA |
| N-35 | 12 | METTTTAQETSAAAVQGTAKVTSSRQELNPLK |
| | | SIVEKSILLTEQALAKAGKGMHGGVPGGKQFIE |
| | | NGSEFAQKLLKKFSLLKPWA |
| N-45 | 13 | MAAAVQGTAKVTSSRQELNPLKSIVEKSILLTE |
| | | QALAKAGKGMHGGVPGGKQFIENGSEFAQKL |
| | | LKKFSLLKPWA |
| N-65 | 14 | MKSIVEKSILLTEQALAKAGKGMHGGVPGGKQ |
| | | FIENGSEFAQKLLKKFSLLKPWA |
| N-71 | 15 | MSILLTEQALAKAGKGMHGGVPGGKQFIENGS |
| | | EFAQKLLKKFSLLKPWA |
| GFP-LAC | | Tri-molecular fusion with Green |
| | | Fluorescence Protein (GFP) fused |
| | | to lacritin and intein as P/O-GFP- |
| | | lacritin-intein. Fluoresces as a |
| | | tri-molecular fusion and as a GFP- |
| | | lacritin fusion. |
| E₂₃→A₂₃ | 16 | MEDASSDSTGADPAQEAGTSKPNAGISGPAEPA |
| E₂₄→G₂₄ | | SPPETTTTAQETSAAAVQGTAKVTSSRQELNPL |
| | | KSIVEKSILLTEQALAKAGKGMHGGVPGGKQFI |
| | | ENGSEFAQKLLKKFSLLKPWA |
| K₈₂→I₈₂ | 17 | MEDASSDSTGADPAQEAGTSKPNEEISGPAEPA |
| K₈₅→I₈₅ | | SPPETTTTAQETSAAAVQGTAKVTSSRQELNPL |
| | | KSIVEKSILLTEQALAIAGIGMHGGVPGGKQFIE |
| | | NGSEFAQKLLKKFSLLKPWA |
| E₂₃→A₂₃ | 18 | MEDASSDSTGADPAQEAGTSKPNAGISGPAEPA |
| E₂₄→G₂₄ | | SPPETTTTAQETSAAAVQGTAKVTSSRQELNPL |
| K₈₂→I₈₂ | | KSIVEKSILLTEQALAIAGIGMHGGVPGGKQFIE |
| K₈₅→I₈₅ | | NGSEFAQKLLKKFSLLKPWA |
| 4Y-Lac | 19 | YYYYEDASSDSTGADPAQEAGTSKPNEEISGPA |
| | | EPASPPETTTTAQETSAAAVQGTAKVTSSRQEL |
| | | NPLKSIVEKSILLTEQALAIAGIGMHGGVPGGK |
| | | QFIENGSEFAQKLLKKFSLLKPWA |
| 4Y/C-5 | 20 | YYYYEDASSDSTGADPAQEAGTSKPNEEISGPA |
| | | EPASPPETTTTAQETSAAAVQGTAKVTSSRQEL |
| | | NPLKSIVEKSILLTEQALAKAGKGMHGGVPGG |
| | | KQFIENGSEFAQKLLKKFSL |
| 4Y/C-10 | 21 | YYYYEDASSDSTGADPAQEAGTSKPNEEISGPA |
| | | EPASPPETTTTAQETSAAAVQGTAKVTSSRQEL |
| | | NPLKSIVEKSILLTEQALAKAGKGMHGGVPGG |
| | | KQFIENGSEFAQKLL |
| 4Y/C-15 | 22 | YYYYEDASSDSTGADPAQEAGTSKPNEEISGPA |
| | | EPASPPETTTTAQETSAAAVQGTAKVTSSRQEL |
| | | NPLKSIVEKSILLTEQALAKAGKGMHGGVPGG |
| | | KQFIENGSEF |
| 4Y/C-20 | 23 | YYYYEDASSDSTGADPAQEAGTSKPNEEISGPA |
| | | EPASPPETTTTAQETSAAAVQGTAKVTSSRQEL |
| | | NPLKSIVEKSILLTEQALAKAGKGMHGGVPGG |
| | | KQFIE |
| 4Y/C-25 | 24 | YYYYEDASSDSTGADPAQEAGTSKPNEEISGPA |
| | | EPASPPETTTTAQETSAAAVQGTAKVTSSRQEL |
| | | NPLKSIVEKSILLTEQALAKAGKGMHGGVPGG |
| 4Y/C-59 | 25 | YYYYEDASSDSTGADPAQEAGTSKPNEEISGPA |
| | | EPASPPETTTTAQETSAAAVQGTAKVTSSRQ |

Other aspects of the invention provide antimicrobial protein variants comprising proteolytically cleaved lacritin. In some embodiments, the antimicrobial lacritin variants are specific for the pathogen that produced the lacritin-cleaving protease. Without wishing to be bound by any particular theory, full-length lacritin may bind to specific human cell surface receptors, initiating a cascade of cell signaling events that culminate in new cell growth. Following this signaling event, the protein may be processed by proteolytic cleavage to produce truncated proteins with new antimicrobial activities. Human tears may contain a set of undiscovered proteases that recognize and cleave lacritin to produce numerous antimicrobial peptide fragments. Some of these proteases may be turned on as a consequence of microbial infection. Certain pathogenic bacteria are known to express proteases and these proteases may cleave lacritin to produce an antimicrobial peptide specific to that pathogen.

Yet other aspects of the invention provide amphipathic antimicrobial lacritin protein variants. Computer analysis of the lacritin protein predicts an amphipathic alpha helix in the C-terminus. Without wishing to be bound by any particular theory, this amphipathic alpha helix may interact with microbial cell membranes, creating channels or pores in the outer membranes of pathogens and resulting in loss of membrane function and cell death.

The present invention is also directed to novel methods, including methods for treating and preventing microbial diseases and infections. The invention can be effective at treating and preventing a broad spectrum of microbial diseases, including but not limited to microbial diseases affecting the eye, such as those described in Levinson and Rutzen, Ophthalmol Clin N Am 18 (2005) 493-509, incorporated herein by reference in its entirety. Infections at sites in the eye or at other sites on the animal can be treated and/or prevented with the methods of the invention.

It is further contemplated that the antimicrobial protein variants of the invention may be used in combination with or to enhance the activity of other antimicrobial agents or antibiotics. Combinations of the peptides with other agents may be useful to allow antibiotics to be used at lower doses due to toxicity concerns, to enhance the activity of antibiotics whose efficacy has been reduced or to effectuate a synergism between the components such that the combination is more effective than the sum of the efficacy of either component independently. Antibiotics which may be combined with an antimicrobial peptide in combination therapy include but are not limited to penicillin, ampicillin, amoxycillin, vancomycin, cycloserine, bacitracin, cephalolsporin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraminosalicylic acid, and ethambutol.

In some embodiments, the methods of the invention comprise administering to a subject a therapeutically effective amount of a recombinant lacritin protein variant. Non-limiting examples of some of the administered recombinant lacritin protein variants in the methods of the invention include the protein variants of Table 1. The antimicrobial proteins can be effective against a number of pathogenic organisms, for example the organisms described in Levinson and Rutzen, Ophthalmol Clin N Am 18 (2005) 493-509. The invention has broad spectrum antimicrobial properties effective against both Gram-positive and Gram-negative strains of bacteria and thus can be effective to multiply drug resistant kill strains.

In one embodiment, the present invention is directed to use of a purified polypeptide comprising an amino acid sequence of a polypeptide of the invention, or a bioactive fragment thereof, or an amino acid sequence that differs by one or more conservative amino acid substitutions. More preferably, the purified polypeptide comprises an amino acid sequence that differs by 20 or less conservative amino acid substitutions, and more preferably by 10 or less conservative amino acid substitutions. Alternatively, the polypeptide may comprise an amino acid sequence that differs by 1 to 5 alterations, wherein the alterations are independently selected from a single amino acid deletion, insertion, or substitution.

In accordance with one embodiment, a method is provided for treating infections of the eye. The method comprises the step of topically administering a composition comprising a lacritin polypeptide to the eye. In one embodiment, the composition further comprises an additional anti-microbial agent. Suitable ophthalmic anti-microbial agents are known to those skilled in the art and include those described in U.S. Pat. Nos. 5,300,296, 6,316,669, 6,365,636 and 6,592,907, the disclosures of which are incorporated herein. Examples of anti-microbial agents suitable for use in accordance with the present invention include benzalkonium chloride, benzethonium chloride, benzyl alcohol, chlorobutanol, chlorhexidine digluconate or diacetate, methyl and propyl hydroxybenzoate (parabens), phenylethyl alcohol, phenylmercuric acetate or nitrate, sorbic acid, and thimerosal.

Recombinant cloning and expression of native lacritin and variants thereof were performed according to methods well known to the skilled artisan and as described, for example, in Sambrook et al., Molecular Cloning: A Laboratory Manual, Cold Springs Harbor Laboratory, 2^{nd} ed., Cold Springs Harbor, N.Y. (1989). Other references describing molecular biology and recombinant DNA techniques include, for example, DNA Cloning 1: Core Techniques, (D. N. Glover, et al., eds., IRL Press, 1995); DNA Cloning 2: Expression Systems, (B. D. Hames, et al., eds., IRL Press, 1995); DNA Cloning 3: A Practical Approach, (D. N. Glover, et al., eds., IRL Press, 1995); DNA Cloning 4: Mammalian Systems, (D. N. Glover, et al., eds., IRL Press, 1995); Oligonucleotide Synthesis (M. J. Gait, ed., IRL Press, 1992); Nucleic Acid Hybridization: A Practical Approach, (S. J. Higgins and B. D. Hames, eds., IRL Press, 1991); Transcription and Translation: A Practical Approach, (S. J. Higgins & B. D. Hames, eds., IRL Press, 1996); R. I. Freshney, Culture of Animal Cells: A Manual of Basic Technique, 4^{th} Edition (Wiley-Liss, 1986); and B. Perbal, A Practical Guide To Molecular Cloning, 2^{nd} Edition, (John Wiley & Sons, 1988); and Current Protocols in Molecular Biology (Ausubel et al., eds., John Wiley & Sons), which is regularly and periodically updated.

Suitable expression vectors according to the invention are, for example, bacterial, yeast, or insect plasmids, wide host range plasmids and vectors derived from combinations of plasmid and phage or virus DNA. Vectors derived from chromosomal DNA are also included. Furthermore, an origin of replication and/or a dominant selection marker can be present in the vector according to the invention. The vectors according to the invention are suitable for transforming, transfecting, or infecting a host cell. Exemplary plasmid vectors for expression include pTYB1 (New England Biolabs, Inc.).

Recombinant DNA constructs encoding the lacritin variants may be expressed in any cells suitable for use as host cells for recombinant DNA expression, including bacterial host cells, yeast and other fungi, or insect cells. Suitable host cells transformed with the DNA constructs can be fermented and subjected to conditions which facilitate the expression of the heterologous DNA, leading to the formation of large quantities of the desired protein.

The present invention also encompasses the use of nucleic acid sequences that encode the lacritin polypeptide, or fragments, homologs, and derivatives thereof. Nucleic acid sequences encoding a lacritin polypeptide, or fragments or homologs thereof, can be inserted into expression vectors and used to transfect cells to express recombinant lacritin in the target cells. In accordance with one embodiment, a nucleic acid comprising a nucleic acid sequence encoding a peptide of the invention is inserted into a eukaryotic expression vector in a manner that operably links the gene sequences to the appropriate regulatory sequences, and lacritin is expressed in a eukaryotic host cell. Suitable eukaryotic host cells and vectors are known to those skilled in the art. In particular, nucleic acid sequences encoding lacritin may be added to a cell or cells in vitro or in vivo using delivery mechanisms such as liposomes, viral based vectors, or microinjection. Accordingly, one aspect of the present invention is directed to transgenic cell lines that contain recombinant genes that express a lacritin polypeptide of the invention.

The present invention is also directed to nucleic acid constructs for expressing heterologous genes under the control of the lacritin gene promoter. In accordance with one embodiment, a nucleic acid construct is provided comprising a nucleic acid sequence encoding a polypeptide of the invention operably linked to a heterologous gene. In accordance with one embodiment, the heterologous gene is a reporter gene that encodes for a marker. The marker can be any gene product that produces a detectable signal and includes proteins capable of emitting light such as Green Fluorescent Protein (GFP) (Chalfie et al., 1994, Science 11: 263:802-805) or luciferase (Gould et al., 1988, Anal. Biochem. 15: 175: 5-13), as well as proteins that can catalyze a substrate (e.g., such as β-galactosidase). The marker may also comprise intracellular or cell surface proteins that are detectable by antibodies. Reporter molecules additionally, or alternatively, can be detected by virtue of a unique nucleic acid sequence not normally contained within the cell.

As used herein, AGFP@ refers to a member of a family of naturally occurring fluorescent proteins, whose fluorescence is primarily in the green region of the spectrum. The term includes mutant forms of the protein with altered or enhanced spectral properties. Some of these mutant forms are described in Cormack, et al., 1996, Gene 173: 33-38 and Ormo, 1996, Science 273:1392-1395, the entireties of which are incorporated herein by reference. The term also includes polypeptide analogs, fragments or derivatives of GFP polypeptides which differ from naturally-occurring forms by the identity or location of one or more amino acid residues, (e.g., by deletion, substitution or insertion) and which share some or all of the properties of the naturally occurring forms so long as they generate detectable signals (e.g., fluorescence). Wild type GFP absorbs maximally at 395 nm and emits at 509 nm. High levels of GFP expression have been obtained in cells ranging from yeast to human cells. The term also includes Blue Fluorescent Protein (BFP), the coding sequence for which is described in Anderson, et al., 1996, Proc. Natl. Acad. Sci. USA 93:16:8508-8511, incorporated herein by reference.

The desired recombinant proteins may be purified prior to administration to a subject. The optional purification procedure for the recombinant proteins present in the host cell extract or culture medium may be based on the properties of the biomolecules, such a size, charge, and function. Methods of purification include centrifugation, electrophoresis, chromatography, dialysis, or a combination thereof. As known in the art, electrophoresis may be utilized to separate the proteins in the sample based on size and charge. Electrophoretic procedures are well known to the skilled artisan, and include isoelectric focusing, sodium dodecyl sulfate polyacrylamide gel electrophoresis (SDS-PAGE), agarose gel electrophoresis, and other known methods of electrophoresis.

The purification step may be accomplished by a chromatographic fractionation technique, including size fractionation, fractionation by charge and fractionation by other properties of the biomolecules being separated. As known in the art, chromatographic systems include a stationary phase and a mobile phase, and the separation is based upon the interaction of the biomolecules to be separated with the different phases. In some forms of the invention, column chromatographic procedures may be utilized. Such procedures include partition chromatography, adsorption chromatography, size-exclusion chromatography, ion-exchange chromatography, and affinity chromatography. Such methods are well known to the skilled artisan. For example, if the desired protein has a known binding affinity domain, such as the heparin binding domains of the chimeric proteins of the present invention, the proteins may be purified by affinity chromatography using heparin agarose columns. An affinity tag may also be engineered into the desired protein for purification purposes. For example, DNA constructs may encode for chitin binding regions attached to the protein variants to facilitate protein purification on chitin affinity columns.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, N.Y. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride, or an “active ester” group such as hydroxybenzotriazole or pentafluorophenly esters.

Examples of solid phase peptide synthesis methods include the BOC method which utilized tert-butyloxcarbonyl as the a-amino protecting group, and the FMOC method which utilizes 9-fluorenylmethyloxcarbonyl to protect the α-amino of the amino acid residues, both methods of which are well known by those of skill in the art.

Incorporation of N— and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N— and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono-and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for homologs of proteins. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on peptide function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The invention also includes a kit comprising the composition of the invention and an instructional material which describes administering the composition to a subject. In another embodiment, this kit comprises a (preferably sterile) solvent suitable for dissolving or suspending the composition of the invention prior to administering the composition.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, and to birds including commercially relevant birds such as chickens, ducks, geese, and turkeys.

Pharmaceutical compositions that are useful in the methods of the invention may be prepared, packaged, or sold in formulations suitable for oral, rectal, vaginal,. parenteral, intravenous, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient,-a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to a subject, preferably a human, range in amount from 1 μg to about 100 g per kilogram of body weight of the subject. While the precise dosage administered will vary depending upon any number of factors, including but not limited to, the type of subject and type of disease state being treated, the age of the subject and the route of administration. Preferably, the dosage of the compound will vary from about 1 mg to about 10 g per kilogram of body weight of the subject. More preferably, the dosage will vary from about 10 mg to about 1 g per kilogram of body weight of the subject.

In one aspect, an effective amount of lacritin and fragments, homologs, derivatives, and modifications thereof is about 0.01 to about 100.0 μg/ml. In one aspect, an effective amount is about 0.1 to about 50 μg/ml. In yet another aspect, an effective is about 0.5 to about 11 μg/ml. In one aspect, the homologs comprise up to about 10 amino acid substitutions. In one aspect, the substitutions are conservative. In another aspect, the homologs comprise up to about 5 amino acid substitutions of lacritin and fragments, homologs, derivatives, and modifications thereof.

The compound may be administered to a subject as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the subject, etc.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a subject. The instructional material of the kit of the invention may, for example, be affixed to a container which contains the peptide of the invention or be shipped together with a container which contains the peptide. Alternatively, the instructional material may be shipped separately from the container with the intention that the instructional material and the compound be used cooperatively by the recipient.

In the context of medical devices, it is envisioned that the protein variants in their pure form or combined with other antimicrobial peptides or agents, could be sprayed on, coated on, or adhered to any surface of a medical device wherein the inhibition of microbial growth on such a surface is desired. Examples of such medical devices include but are not limited to tubes, catheters, stents, valves, implants, and other devices.

It is further contemplated that the antimicrobial protein variants of the invention may be used in combination with or to enhance the activity of other antimicrobial agents or antibiotics. Combinations of the peptides with other agents may be useful to allow antibiotics to be used at lower doses due to toxicity concerns, to enhance the activity of antibiotics whose efficacy has been reduced or to effectuate a synergism between the components such that the combination is more effective than the sum of the efficacy of either component independently. Antibiotics which may be combined with an antimicrobial peptide in combination therapy include but are not limited to penicillin, ampicillin, amoxycillin, vancomycin, cycloserine, bacitracin, cephalolsporin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraminosalicylic acid, and ethambutol.

Further embodiments of the invention include therapeutic kits that comprise, in suitable container means, a pharmaceutical formulation of at least one antimicrobial lacritin variant. Some embodiments provide kits comprising a pharmaceutical formulation comprising at least one antimicrobial lacritin variant and a pharmaceutical formulation of at least one antimicrobial agent or antibiotic. The antimicrobial peptide and antimicrobial agent or antibiotic may be contained within a single container means, or a plurality of distinct containers may be employed.

Reference will now be made to specific examples illustrating the constructs and methods above. It is to be understood that the examples are provided to illustrate preferred embodiments and that no limitation to the scope of the invention is intended thereby.

### Examples

Materials and Methods

Lacritin Purification. For protein expression, cultures of E. coli strain ER2566 harboring the plasmid of interest were grown at 37° C. to mid-log and the temperature reduced to 23° C. to minimize secretion in inclusion bodies prior to induction. The cultures were induced with 0.5 mM isopropyl-beta-D-thiogalactopyranoside (IPTG) for 4 h at 23° C. Cells were harvested by centrifugation and stored at −70° C. Frozen cell pellets were thawed at room temperature, lysed by sonication in 50 mM Tris (pH 8), 0.5 M NaCl, 0.45% Triton X-100 and centrifuged. The cleared supernatant was loaded onto a chitin column (IMPACT-CN System; New England Biolabs Inc., Beverly Mass.) equilibrated with 10 column volumes of 50 mM Tris (pH 8), 0.5 M NaCl and washed with twenty column volumes of the same buffer. On-column cleavage of lacritin from C-terminal intein was accomplished by incubation for 16 hours at room temperature with 50 mM dithiothreitol in the same buffer. Eluates were concentrated, dialyzed extensively against PBS (4° C.). The dialyzed chitin fraction was loaded onto a DEAE Sepharose Fast Flow column equilibrated with 10 column volumes of PBS. The flow through unbound fraction was collected, assayed for protein concentration, aliquoted, and stored at −80 degrees C.

Antimicrobial Assays. The antimicrobial activities were tested using a colony forming unit assay. E. coli (ATCC #10536) was grown in LB medium at 37° C. to an OD600 of 0.5. The cells were washed 3 times in phosphate buffer (pH 7.2) and resuspended in 1 ml of the same buffer. The cells were then diluted 1:100 using two 10 fold serial dilutions. Following dilutions, 50 μl of cells were incubated with 100 μl of sample protein and 350 μl of phosphate buffer at 37° C. In place of sample protein, 100 μl of 10 mM phosphate buffered saline (pH 7.4) was used as the control sample. Following incubation, the cells were diluted 1:10 before plating 100 μl-200 μl in quadruplicates. The plates were incubated overnight at 37° C. and colony forming units were counted. The percentage of cell death was expressed using the following equation, [1-(cells surviving peptide)/(average colonies counted from PBS control)]×100%. The LC₅₀ is defined as the lethal concentration of peptide required to kill 50 percent of the present bacteria and was determined for each Lacritin variant by extrapolation of concentration curves.

Inner Membrane Permeabilization. To examine the affect of Lacritin constructs on the inner membrane of gram negative bacteria, an enzymatic assay was conducted using a strain of permease-deficient E. coli, designated E. coli ML-35 (ATCC #43827) which also constitutively produces cytoplasmic βeta-galactosidase. E. coli ML-35 were grown in LB medium for 20 hours at 37° C. as a saturated overnight and then diluted with LB to an absorbance OD600 of 0.5. The cells were washed 3 times in 10 mM sodium phosphate buffer-10 mM NaCl (pH 7.2) and resuspended in 1 ml of the same buffer. The cells were then diluted 1:100 in 10 mM sodium phosphate buffer using two serial 10-fold dilutions. 70 μl of phosphate buffer, 30 μl of 30 mM βeta-galactosidase substrate ONPG (o-nitrophenyl-β-D-galactopyranoside) in 20 mM sodium phosphate buffer, 100 μl of cells in 10 mM sodium phosphate buffer and 50 μl of peptide sample were added to microtiter plate wells in duplicate. The production of ONP (o-nitrophenyl) from ONPG was measured over time using a spectrophotometer set at a wavelength of 415 nm.

### Example 1

### Cloning and Expression of Lacritin Variants

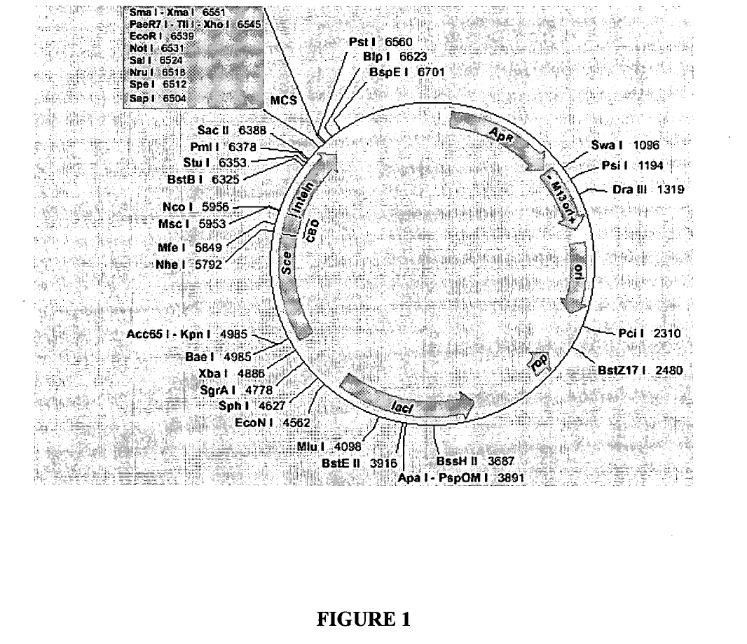

The variant lacritin sequences were cloned into a bacterial expression vector and sequenced to confirm that the expected DNA coding sequences had been successfully cloned. FIG. 1 shows a diagram of this vector and identifies the relevant genetic information. Cloned lacritin variants were transformed into E. coli strain ER2566, frozen, stored at −70° C., and used as starting material for the production of recombinant proteins. Frozen cells containing the construct of interest were cultured in LB media at 37° C. and induced for expression of the variant lacritin gene by addition of the chemical IPTG (isopropyl-beta-D-thiogalactopyranoside).

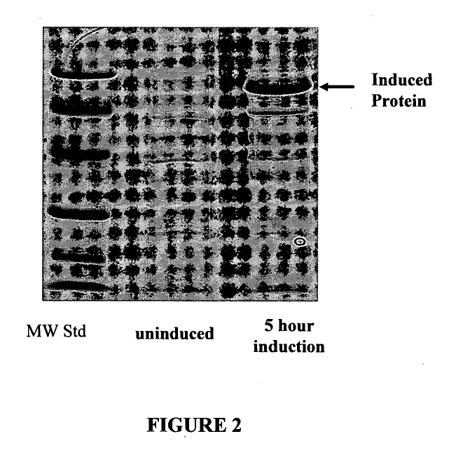

FIG. 2 shows a protein profile of cell extracts from uninduced cells and cells after five hours of induction with IPTG. An induced protein band of the expected molecular weight is clearly visible demonstrating that that this expression system is producing recombinant proteins upon induction. Following induction, cells were harvested by centrifugation and lysed by sonication. Crude cell extracts were loaded onto a chitin column, washed to remove unbound proteins, and subjected to reducing conditions to catalyze on-column cleavage of the recombinant proteins. Eluted proteins were concentrated by ultrafiltration, dialyzed extensively against phosphate buffered saline (PBS) and stored at −70° C. for later analysis.

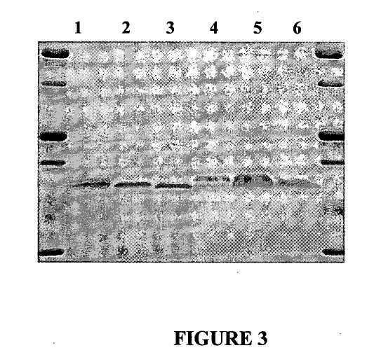

FIG. 3 shows examples of purified recombinant lacritin protein variants visualized by SDS Polyacrylamide Gel Electrophoresis (PAGE) stained with Coomassie blue.

### Example 2

### Antimicrobial Activity Assay

Purified recombinant lacritin variant proteins were assayed for antimicrobial activity by standard methods that involves an incubation period with actively growing bacteria and the protein of interest followed by plating on agar plates supplemented with growth media. The agar plates are incubated overnight and individual colonies are counted and expressed as Colony Forming Units (CFUs). The number of CFUs can be compared to the same bacteria incubated without protein.

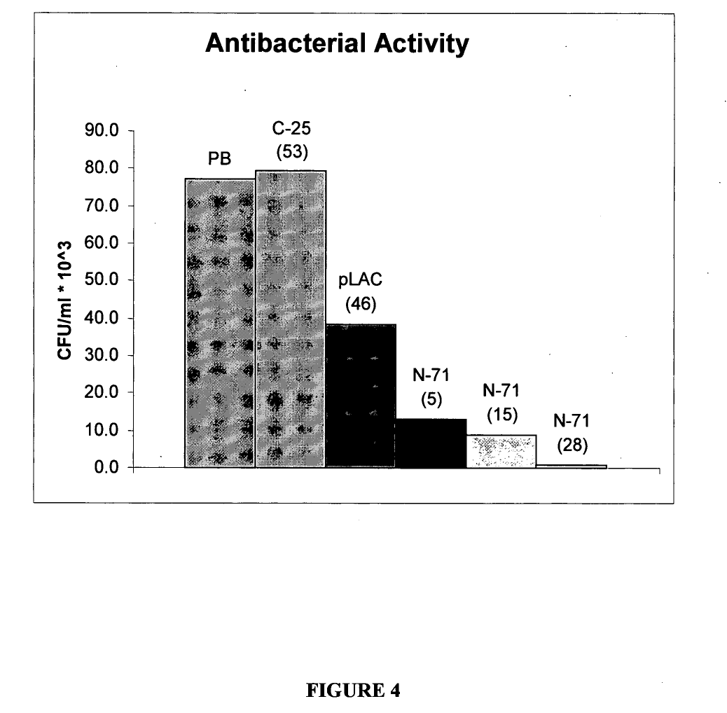

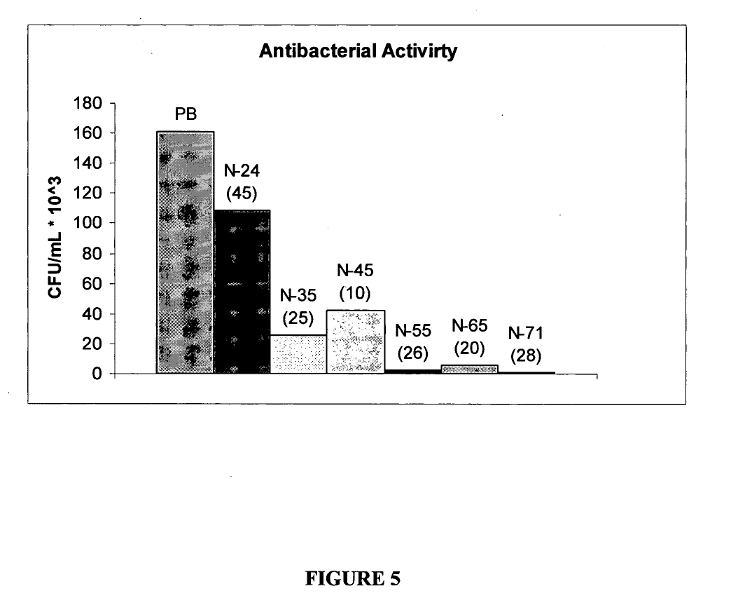

FIGS. 4 and 5 shows examples of an antimicrobial experiment in which a number of different lacritin variant proteins were incubated with the bacteria E. coli and then plated for colony forming units. E. coli at exponential growth (OD₆₀₀=0.5) were washed twice in Phosphate Buffer (PB) diluted to a concentration of 1×10⁶ cells/ml in PB and incubated with PB (no protein) or test proteins at 37° C. Samples were diluted and plated in triplicate on LB plates after four hours of incubation. Plates were grown at 37° C. overnight and counted. Colony counts were averaged and plotted as CFU/ml×10³. The incubation volume was 0.5 ml at 25 mM NaCl.

FIG. 4 shows the results of an antimicrobial experiment for the lacritin variants C-25 and N-71 (the number following the “N—” refers to the number of amino acids removed from the N-terminus, and the number following the “C—” refers to the number of amino acids removed from the C-terminus, of full-length mature lacritin). C-25 was tested at a concentration of 53 μg/ml and N-71 was tested at various concentrations in μg/ml as shown in parentheses. pLAC is full length recombinant mature lacritin, and was tested at a concentration of 46 μg/ml. Bacteria incubated with buffer alone (the control) produced an average of 77×10³ CFU/ml. Incubation with the lacritin variant C-25 produced an average of 79×10³ or 103% of the control. Clearly, the variant protein C-25 had no affect on bacterial viability. The full-length recombinant lacritin protein (PLAC) produced an average of 38×10³ CFU/ml or 49% of the control. The variant protein N-71 produced a concentration dependent antimicrobial activity that was less than 1% of the control at a concentration of 28 μg/ml. These data show that coding sequences within the lacritin gene can produce a variant protein capable of killing greater than 99% of E. coli cells in a controlled experiment.

FIG. 5 shows the results of an antimicrobial experiment for the lacritin variants N-24, N-35, N-45, N-55, N-65 and N-71 (the number following the “N—” refers to the number of amino acids removed from the N-terminus of full-length mature lacritin). The variant lacritin proteins were tested at the concentrations shown in parentheses in μg/ml. Bacteria incubated with buffer alone (the PB control) produced an average of 161×10³ CFU/ml. Incubation with the lacritin variant N-24 produced an average of 108×10³ or 63% of the control. The variant protein N-24 had little or no affect on bacterial viability. The variant protein N-35 produced an average of 25×10³ CFU/ml or 16% of the control and the variant N-45 produced an average of 42×10³ CFU/ml or 26% of the control. These variants are active in antimicrobial activity killing 84% and 74% respectively of the bacteria under the conditions tested. The variant N-55 produced an average of 2×10³ CPU/ml or 0.01% of the control, the variant N-65 produced an average of 6×10³ CFU/ml or 0.04% of the control, and the variant N-71 produced an average of 1×10³ CFU/ml or 0.01% of the control. These data show that coding sequences within the lacritin gene can produce variant proteins capable of killing greater than 99% of E. coli cells in a controlled experiment.

### Example 3

### Further Studies

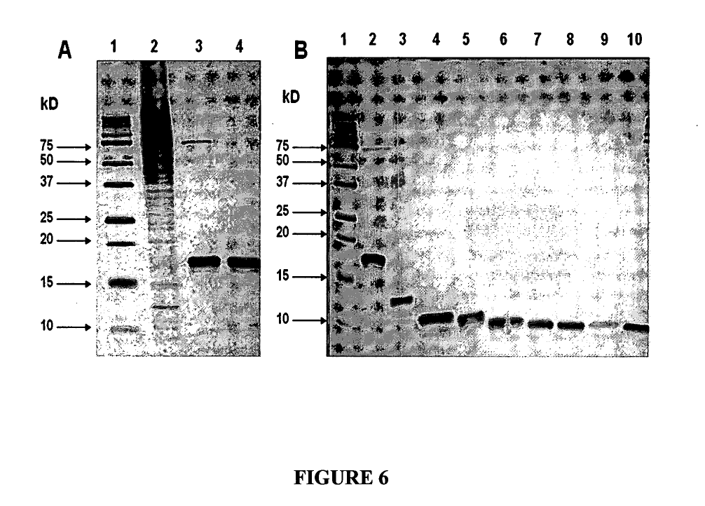

FIG. 6 shows the results of SDS Polyacrylamide Gel Electrophoresis of Lacritin Proteins. A. Purification of mature lacritin. Lanes (1) molecular weight markers labeled in kilodaltons (2) fraction 1, cleared cell lysate (3) fraction 2, chitin purified (4) fraction 3, DEAE purified. B. DEAE purified lacritin proteins. Lanes (1) molecular weight markers labeled in kilodaltons (2) mature lacritin (3) N-35 (4) N-45 (5) N-55 (6) N-65 (7) N-71 (8) N-72 (9) N-73 (10) N-75. N-XX denotes the number of amino acids removed from the N-terminal of mature lacritin. 15% acrylamide gels from BioRad were run at 140 volts and silver stained.

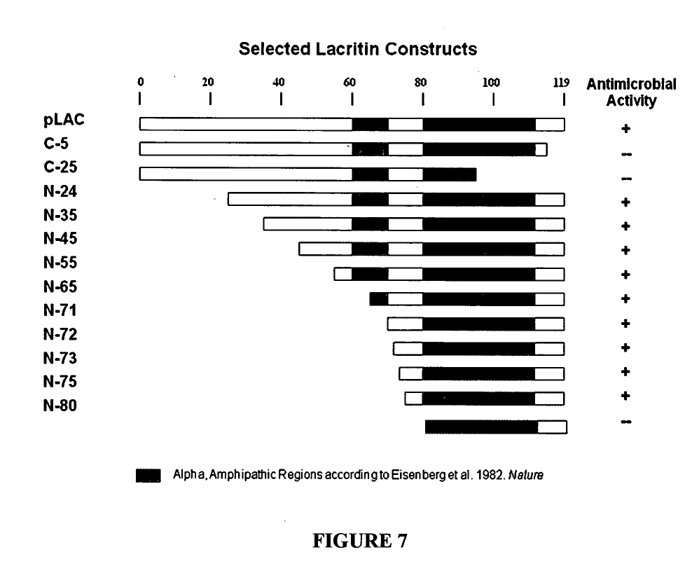

FIG. 7 shows the results of experiments testing the antimicrobial activity of selected lacritin constructs. pLAC is full length mature lacritin (SEQ ID NO:27) without signal peptide. N-XX denotes the number of amino acids removed from the N-terminal of mature lacritin and C-XX denotes the number of amino acids removed from the C-terminal of mature lacritin. The numbers 0 through 119 are the amino acid residues of mature lacritin from the C-terminus to the N-terminus.

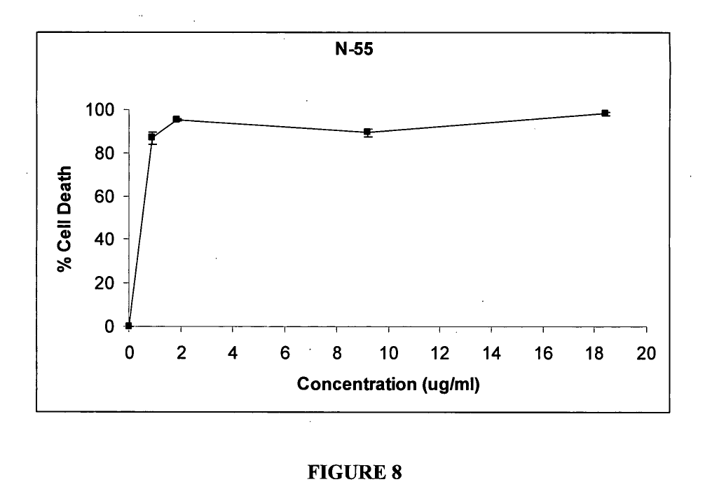

FIG. 8 shows the results of experiments testing the antimicrobial activity of lacritin N-55. Increasing concentrations of the purified protein were incubated with E. coli in 10 mM phosphate buffer for 3 hours as described in the antimicrobial assay. The total number of colonies were counted and the percent of cell death determined using [1-(colonies surviving peptide incubation)/(colony count from PBS control)]×100.

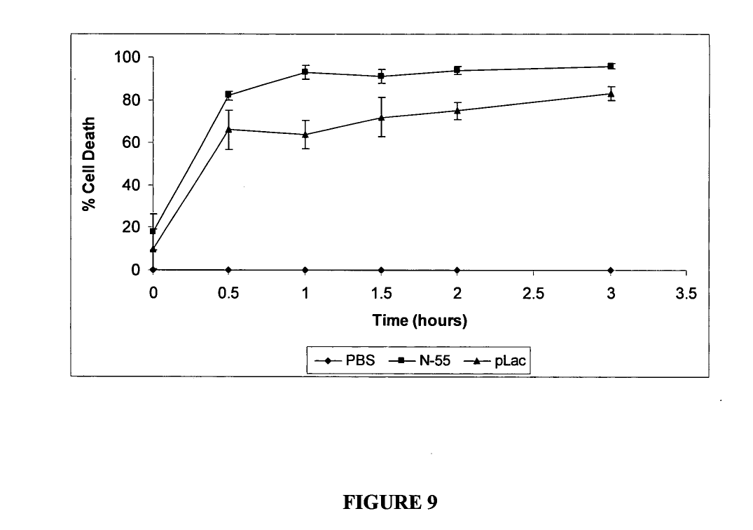

FIG. 9 shows the results of experiments testing a time course of antimicrobial activity of Lacritin. Lacritin construct N-55 (▪ 30 μg/ml), mature Lacritin (▴ 30 μg/ml) and the phosphate buffered saline control () were incubated with E. coli from 0 to 3 hours as described in the antimicrobial assay. Colonies were counted and the percent cell death was determined using [1-(cells surviving peptide)/(average colonies counted from PBS control)]×100%.

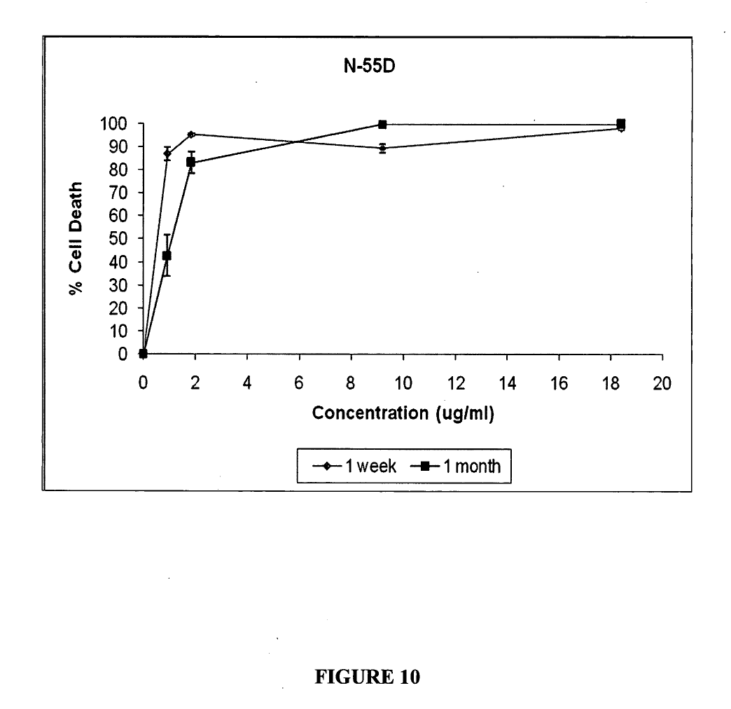

FIG. 10 shows the results of experiments testing the stability of lacritin N-55 antimicrobial activity. Increasing concentrations of the purified protein were incubated with E. coli in 10 mM phosphate buffer for 3 hours as described in the antimicrobial assay. Aliquots of lacritin were stored at −70 degrees C. and assayed after one week and one month of storage as shown. The total number of colonies were counted and the percent of cell death determined using [1-(colonies surviving peptide incubation)/(colony count from PBS control)]×100.

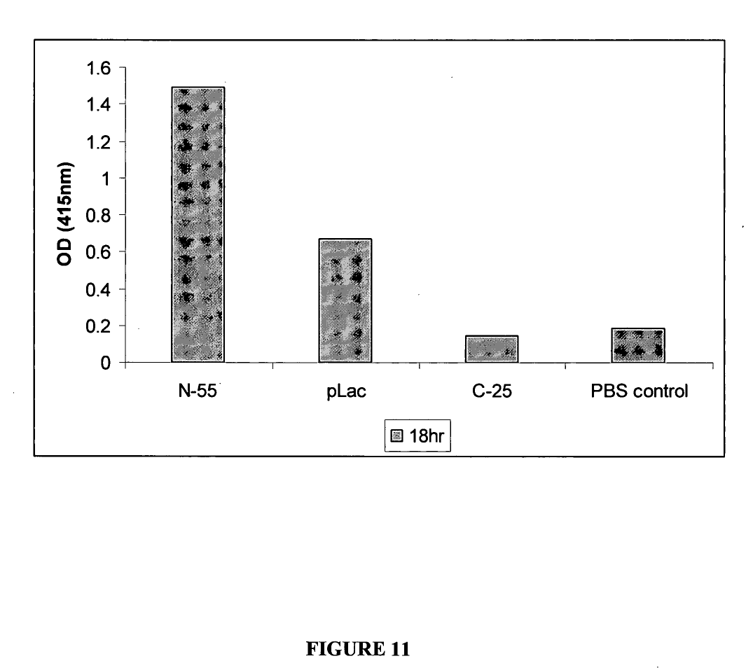

FIG. 11 shows the results of experiments testing the inner membrane permeabilization of E. coli ML-35. Mature Lacritin (pLac) and various constructs of Lacritin were incubated with permease-deficient E. coli ML-35 at room temperature for 18 hours. The conversion of ONPG (o-nitrophenyl-β-D-galactopyranoside) to ONP (o-nitrophenyl) by cytoplasmic β-galactosidase was measured by a spectrophotometer at 415 nm. The concentrations of each protein were normalized to 30 μg/ml. This enzymatic assay indicates that both the mature antimicrobial peptide Lacritin and the construct N-55 damage the cell membrane in such a way that cytoplasmic β-galactosidase is able to react with the surrounding ONPG substrate.

Table 2 demonstrates the LC₅₀ antimicrobial activity of selected lacritin constructs. Lethal concentrations (μg/ml) of lacritin constructs required to kill 50 percent of E. coli in the antimicrobial assay. Lacritin is full length mature lacritin without signal peptide. N-XX denotes the number of amino acids removed from the N-terminal of mature lacritin and C-XX denotes the number of amino acids removed from the C-terminal of mature lacritin.

TABLE 2
LC₅₀ antimicrobial activity of selected lacritin constructs.

| Lacritin | LC₅₀ |
|---|---|
| Construct | (μg/ml) |
| Lacritin | 11.0 |
| N-35 | 1.5 |
| N-45 | 1.0 |
| N-55 | 0.5 |
| N-65 | 5.5 |
| N-71 | 8.0 |
| N-72 | 7.5 |
| N-73 | 8.0 |
| N-80 | Not Active |
| C-25 | Not Active |

Table 3 demonstrates the results of experiments testing the antimicrobial activity of lacritin variants against selected pathogenic bacteria. Purified lacritin variant proteins at the concentrations shown were incubated with selected pathogenic bacteria in 10 mM phosphate buffer for 3 hours as described in the antimicrobial assay. The total number of colonies were counted and the percent cell death determined using [1-(colonies surviving peptide incubation)/(colony count from PBS control)]×100. N-XX denotes the number of amino acids removed from the N-terminal of mature lacritin.

TABLE 3
Antimicrobial activity of lacritin against selected pathogenic bacteria

| | Lacritin | Cell Death | Concentration |
|---|---|---|---|
| Organism | Construct | % | (μg/ml) |
| Pseudomonas aeruginosa | N-65 | 75 | 19.2 |
| Pseudomonas aeruginosa | N-55 | 100 | 23.0 |
| Pseudomonas aeruginosa | N-45 | 99 | 23.0 |
| Pseudomonas aeruginosa | N-35 | 95 | 36.8 |
| Staphylococcus aureus | N-55 | 58 | 23.3 |
| Staphylococcus aureus | N-45 | 58 | 26.3 |
| Staphylococcus epidermidis | N-35 | 69 | 37.6 |
| Staphylococcus epidermidis | N-65 | 33 | 21.6 |

Headings are included herein for reference and to aid in locating certain sections. These headings are not intended to limit the scope of the concepts described therein under, and these concepts may have applicability in other sections throughout the entire specification.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An antimicrobial composition for treating or preventing a microbial infection, said composition comprising at least one lacritin polypeptide, or a fragment, homolog, derivative, or modification thereof, a pharmaceutically acceptable carrier, and optionally another antimicrobial agent.

2. The antimicrobial composition of claim 1, wherein said at least one lacritin polypeptide, or a fragment, homolog, derivative, or modification thereof comprises an amino acid sequence selected from the group consisting of SEQ ID NOs:1, 2, 3, 4, 5, 6, 7, 8, 9, 10, 11, 12, 13, 14, 15, 16, 17, 18, 19, 20, 21, 22, 23, 24, 25, 26, and 27.

3. An antimicrobial composition comprising at least one isolated nucleic acid, wherein said at least one isolated nucleic acid comprises a nucleic acid sequence encoding a lacritin polypeptide, or a fragment or homolog thereof, a pharmaceutically acceptable carrier, and optionally another antimicrobial agent.

4. A method of treating or preventing a microbial infection, said method comprising administering to a subject in need thereof a pharmaceutical composition comprising a pharmaceutically acceptable carrier, a therapeutically effective amount of at least one lacritin polypeptide, or a fragment, homolog, derivative, or modification thereof, and optionally another antimicrobial agent.

5. The method of claim 4, wherein said at least one lacritin polypeptide, or a fragment, homolog, derivative, or modification thereof comprises an amino acid sequence selected from the group consisting of SEQ ID NOs:1, 2, 3, 4, 5, 6, 7, 8, 9, 10, 11, 12, 13, 14, 15, 16, 17, 18, 19, 20, 21, 22, 23, 24, 25, 26, and 27.

6. The method of claim 4, wherein said subject is a human.

7. The method of claim 6, wherein said microbial infection is selected from the group consisting of a bacterial infection, a fungal infection, a viral infection, and a parasitic infection.

8. The method of claim 7, wherein said infection is a bacterial infection.

9. The method of claim 4, wherein said pharmaceutical composition is administered after said subject has been subjected to a microbe.

10. The method of claim 4, further wherein said composition further comprises a therapeutic agent.

11. The method of claim 4, wherein said at least one lacritin polypeptide, or a fragment, homolog, derivative, or modification thereof, is administered at a dose of about 0.1 μg/ml to about 20 μg/ml.

12. The method of claim 4, wherein said composition is administered at least twice.

13. The method of claim 4, wherein said bacteria are selected from the group consisting of Escherichia coli, Pseudomonas aeruginosa, Staphylococcus aureus, and Staphylococcus epidermidis.

14. A method for inhibiting microbial growth, said method comprising contacting microbes with a composition comprising an effective amount of at least one lacritin polypeptide, or a fragment, homolog, derivative, or modification thereof, and optionally another antimicrobial agent.

15. The method of claim 14, wherein said at least one lacritin polypeptide, or a fragment, homolog, derivative, or modification thereof comprises an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, and 27.

16. The method of claim 14, wherein said at least one lacritin polypeptide, or a fragment, homolog, derivative, or modification thereof, is administered at a dose of about 0.1 μg/ml to about 20 μg/ml.

17. A kit for administering a pharmaceutical composition for treating or preventing a microbial infection, said kit comprising a pharmaceutical composition comprising a therapeutically effective amount of at least one lacritin polypeptide, or a fragment, homolog, derivative, or modification thereof, a pharmaceutically acceptable carrier, optionally another antimicrobial or therapeutic agent, an applicator, and an instructional material for the use thereof.
